(19) United States
(12) Patent Application Publication
(10) Pub. No.: US 2009/0136111 A1
(43) Pub. Date: May 28, 2009

(54) SYSTEM AND METHOD OF DIAGNOSING A MEDICAL CONDITION

(75) Inventors: Kadri Nizar Jabri, Waukesha, WI (US); Renuka Uppaluri, Pewaukee, WI (US); Yan Laura Lin, Beijing (CN); Huanzhong Li, Beijing (CN); Gopal Biligeri Avinash, Menomonee Falls, WI (US); John Michael Sabol, Sussex, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/944,620

(22) Filed: Nov. 25, 2007

(51) Int. Cl. A61B 6/00

(52) U.S. Cl. 382/132

(57) ABSTRACT

A system and method of diagnosing a medical condition in a patient from accessing image data and non-image data of a patient, analyzing the combination of image data and non-image data to generate an output with image findings and a risk assessment for diagnosing certain medical conditions in the patient. The image data may be acquired from an image acquisition system. The non-image data may include clinical data of the patient and may be acquired from a user interface, an electronic medical record, and/or findings from an expert system from previous imaging sessions.

### BACKGROUND OF THE INVENTION

This disclosure relates generally to imaging systems and methods, and more particularly to a system and method of more efficiently and accurately diagnosing a medical condition, such as tuberculosis.

Medical imaging, particularly diagnostic imaging, has become a cornerstone of medical practice in all fields. Such imaging has largely displaced interventional processes such as exploratory surgery, and has greatly enhanced the ability to detect and diagnose disease states, and to treat many different medical conditions. A range of diagnostic imaging modalities are currently available, including magnetic resonance (MR), computed tomography (CT), ultrasound, X-ray, positron emission tomography (PET), and others, as well as combinations thereof. In many instances, more than one of these imaging modalities may be key to understanding development of disorders in particular tissues of a patient, useful in performing accurate diagnosis and, ultimately, in rendering high quality medical care.

To improve the efficiency and accuracy of diagnosing certain medical conditions, improved techniques for integrating image data with non-image data and analyzing this combination of data are needed.

Tuberculosis is an example of a medical condition that is in need of more efficient and improved diagnostic accuracy. Tuberculosis kills almost 3 million people per year, more than any other infectious agent, and the current rate of infection is one person per second. It is the leading cause of death among people with HIV and AIDS. Although tuberculosis is treatable, diagnosis is lengthy and awkward.

One of the common diagnostic screening tools for tuberculosis is the standard chest X-ray radiograph. Although the chest X-ray radiograph is sensitive to many abnormalities that may indicate tuberculosis, it is not diagnostically specific enough, and the examining physician usually relies on a wide array of non-image clinical information to assess the risk of a patient having active tuberculosis disease. However, this assessment varies in quality and accuracy due to the large number of radiographic patterns or findings that can be present in a chest X-ray radiograph of a patient currently or previously infected with tuberculosis, the large amount and subjective nature of non-image clinical information and its interpretation, and the history of other active or previous disease that creates radiographic patterns that can mimic or mask the presence of tuberculosis.

Tuberculosis screening is routine in many countries and regions including pre-employment screening, and entry and exit border screening. This generates a huge number of cases and presents a significant workload and potential burden on local healthcare resources. Therefore, efficient screening and rapid processing of these cases is needed. Also, there is a need to effectively register, track and monitor the people screened so that both high-risk and low-risk individuals can be identified, and treatment or follow-up can be monitored.

Therefore, there is a need for a system and method of improving the diagnostic accuracy of medical conditions by assisting the physician in analyzing the combination of a wide variety of image data and non-image clinical data for each patient.

### BRIEF DESCRIPTION OF THE INVENTION

In an embodiment, a system of diagnosing a medical condition in a patient, the system comprising an input of image data of the patient; an input of non-image data of the patient; an expert system for analyzing the image data and the non-image data to determine the prevalence of patterns in the image data and the non-image data; and an output of the analysis results and an assessment of the risk of the medical condition in the patient.

In an embodiment, a computer implemented method of diagnosing a medical condition in a patient, the method comprising accessing image data of the patient generated by an image acquisition system; accessing non-image data of the patient; analyzing the image data and the non-image data to determine the prevalence of pre-determined patterns of interest in the image data and the non-image data; and presenting analysis results of the image data and a risk assessment of the medical condition in the patient to a user for determining a diagnosis.

In an embodiment, a computer implemented method of diagnosing a medical condition in a patient based on an electronic medical record, the method comprising accessing diagnostic image data from the electronic medical record of the patient; accessing non-image data from the electronic medical record of the patient; analyzing the image data and the non-image data to determine the prevalence of pre-determined patterns of interest in the image data and the non-image data; and presenting analysis results of the image data and an assessment of the risk of the medical condition in the patient to a user for determining a diagnosis.

In an embodiment, a computer-readable storage medium having a set of instructions stored thereon for execution by a computer, the set of instructions comprising a routine for accessing image data; a routine for accessing non-image data; a routine for analyzing the image data and the non-image data; and a routine for visualizing results of the analysis of the image data and the non-image data.

Various other features, aspects, and advantages will be made apparent to those skilled in the art from the accompanying drawings and detailed description thereof.

### BRIEF DESCRIPTION OF THE DRAWINGS

FIG. 1 is a block diagram of an exemplary embodiment of a system of diagnosing a medical condition in a patient;

FIG. 2 is a block diagram of an exemplary embodiment of a system of diagnosing a medical condition in a patient;

FIG. 3 is a flow diagram of an exemplary embodiment of a method of diagnosing a medical condition in a patient;

FIG. 4A is a schematic diagram of an exemplary embodiment of an output of a system and method of diagnosing a medical condition in a patient; and

FIG. 4B is a schematic diagram of an exemplary embodiment of an output of a system and method of diagnosing a medical condition in a patient.

### DETAILED DESCRIPTION OF THE INVENTION

Medical professionals desiring to make certain diagnoses or to rule out diagnoses may utilize an expert system implemented through software to evaluate known image information and to draw upon information from an electronic medical record (EMR) to determine the most useful next steps in providing medical care to a patient.

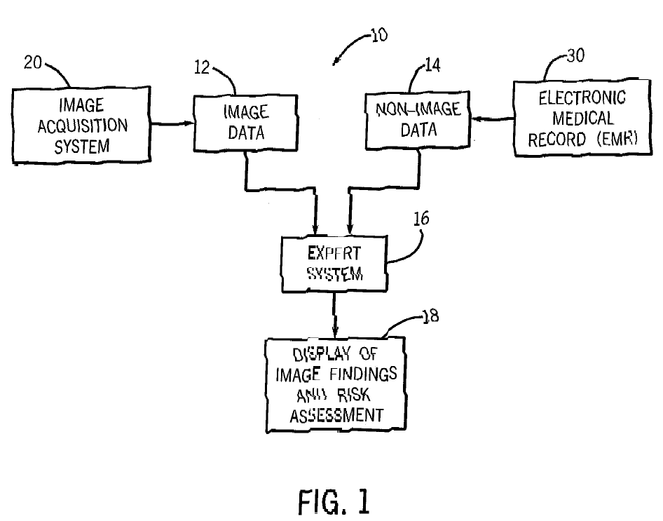

Referring now to the drawings, FIG. 1 illustrates a block diagram of an exemplary embodiment of a system 10 of diagnosing a medical condition in a patient. The system 10 includes an input of image data 12 of a patient acquired from an image acquisition system 20 and an input of non-image clinical data of the patient from an electronic medical record (EMR), both of which are input into an expert system 16 for analyzing the image data 12 and the non-image data 14 to determine patterns in the image data and the non-image data. In an exemplary embodiment, the image acquisition system 20 may be an X-ray system providing image data of X-ray radiographs of a patient. The expert system 16 providing an output 18 display of enhanced image findings and a risk assessment for diagnosing certain medical conditions. The risk assessment is based on both the image data 12 and the non-image data 14. In an exemplary embodiment, the enhanced image findings may be computer aided diagnosis or computer aided detection (CAD) image findings. In an exemplary embodiment, the output 18 may optionally recommend next and/or follow-up steps for proceeding with patient care for a particular patient.

The expert system 16 provides for automatic knowledge-based analysis of image and non-image information. The expert system 16 combines knowledge-based analysis for detecting X-ray radiographic image patterns and non-image data 14. The expert system 16 detects patterns in the non-image data 14 and detects correlations between features in the image data 12 and the non-image data 14.

In an exemplary embodiment, the expert system 16 may be implemented through software, hardware, or a combination thereof. In an exemplary embodiment, the expert system 16 may be integrated into the image acquisition system 16. In an exemplary embodiment, the expert system 16 may be a stand-alone system. In an exemplary embodiment, the expert system 16 may be a fully automated system. In an exemplary embodiment, the expert system 16 may be an on-demand system that may be configurable through a user interface 24.

In an exemplary embodiment, the expert system 16 may include combinations of automated image segmentation algorithms that detect the location, shape, and contour of certain anatomical features; automated X-ray radiograph image pattern detection and classification using pattern recognition algorithms and a knowledge base of medical condition and non-medical condition radiographic findings; a rule-based or learning-based system (e.g., neural networks, support vector machines, genetic algorithms, combinations thereof); a statistical based system (e.g., Bayesian, maximum likelihood, maximum entropy). In an exemplary embodiment, the expert system 16 may use the non-imaging data 14 to customize parameter selections for image-based analysis algorithms.

In an exemplary embodiment, the EMR may include non-imaging data for each individual patient. Steps for building, modifying, and updating the EMR include acquiring the non-image data. The non-image patient data in the EMR may be acquired in any suitable manner, including those used for generating conventional electronic medical records. For example, data may be entered manually or transmitted through wired or wireless communications links and/or networks. The data in the EMR may be updated as new data becomes available. In general, EMR data acquisition is achieved by digitizing or summarizing the data in a manner that permits it to be stored in a computer-readable medium.

In an exemplary embodiment, the non-image data may include the patient's medical history, symptoms, clinical examination results, test results, risk factors, exposure to infectious medical conditions, physiological data, histopathological data, genetic data, pharmacokinetic data, or any combination thereof.

In an exemplary embodiment, the non-image data may include tables containing test results, textual reports of test results (structured and unstructured), and results represented as waveforms pertaining to clinical tests. The non-image data may be compared to known standards or adhoc standards established based on normal (with respect to the clinical condition of interest) people. Patterns of interest may be derived from a plurality of tests for a medical condition.

Examples of preparing non-image data for pattern analysis may be described as follows.

Transformation to standardized/normalized data values: A well-defined normal cohort is used to create the normal's database. The set of normal cohort under go clinical tests corresponding to the clinical case of interest. In the standardized space each test value is assigned a mean value and associated standard deviation based on the data samples from the cohort of normal cases.

Calculation of a deviation value from normal: A method for determining the deviation from normal data values. Each patient's clinical test data is standardized and compared with the normal database's mean value using the following equation:

$$Δ {a}_{i} = \frac{{a}_{i} - {μ}_{{a}_{i}}}{{σ}_{{a}_{i}}}$$

where αᵢ is the i^{th} clinical test of clinical condition α and σₐ and σₐ. This process is applied to all the clinical tests in all the clinical conditions and the resultant is a deviation non-imaging data (metadata) “vector”.

Visualization and display of the deviation data: Deviation from a plurality of clinical tests is a deviation map represented as a synthetic image, where each pixel value is represented by the deviation of a specific clinical test. Patterns can be analyzed from this deviation map.

The image data 12 and non-image data 14 may be transmitted to the expert system 16 through a wired or wireless communications interface. The EMR 30 may be coupled to the expert system 16 through a wired or wireless communications interface using a local area network (LAN) or a wide area network (WAN). The wireless communications interface may be implemented through a wireless communications protocol.

As an example of the above system for diagnosing tuberculosis, the image data input is chest X-ray radiographs of a patient, and the non-image data is clinical information of the patient. In an exemplary embodiment, the chest X-ray radiographs may be digitally created through direct digital radiography (DDR), computed radiography (CR), or a digitized X-ray film. In an exemplary embodiment, the image acquisition system may use a dual-energy exam or a tomosynthesis exam where more than one image is acquired from one or more views for creating the image data. In the case of a single energy image, the chest X-ray radiographs may be a posterior-anterior (PA) view only, or it may be a PA view and a lateral view, or additional views. The image data may also include prior chest X-ray radiographs that were acquired prior to a current imaging session. The non-image data may include the patient's history (including previous expert system results); symptoms (e.g., cough, body temperature); results of blood, sputum and biopsy testing (past or present); exposure to tuberculosis; risk factors for tuberculosis (e.g., recent travel to high-risk regions); exposure to other pathologies that mimic tuberculosis or can change the radiographic appearance of tuberculosis (e.g., HIV) etc. The non-image data can be manually entered through a user interface, directly obtained from EMRs, or updated from a previous expert system analysis of patient data. The system improves access to tuberculosis screening in remote regions of the world where expert physicians may not be available or common. The system also improves tuberculosis screening workflow by enabling on-demand remote tuberculosis diagnosis, increasing review efficiency, and decreasing the burden of high-volume screening.

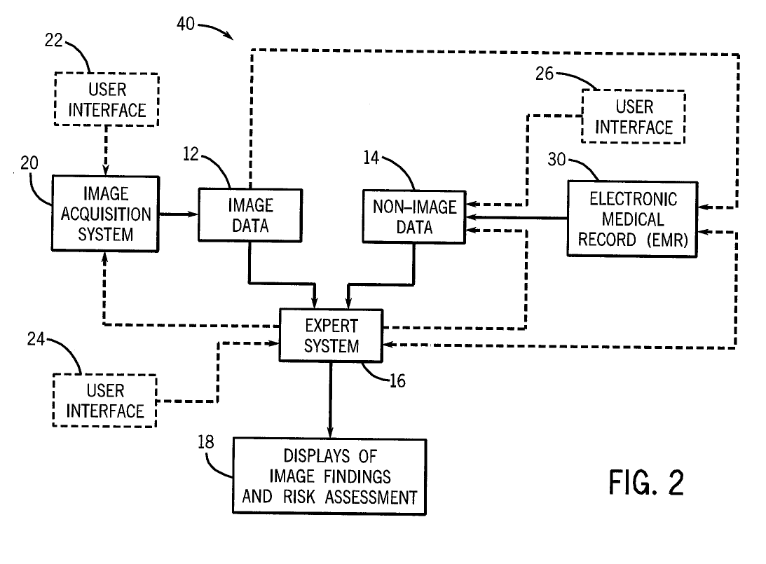

FIG. 2 illustrates a block diagram of an exemplary embodiment of a system 10 of diagnosing a medical condition in a patient. The system 10 includes an image acquisition workstation 20 providing image data 12 to an expert system 16 and/or an EMR 30, a user interface 26 providing non-image data 14 to the expert system 16, the EMR providing non-image data 14 and/or image data 12 to the expert system 16. The non-image data 14 may be manually entered through a user interface 26, directly obtained from the EMR 30, or updated from a previous expert system analysis of patient data. The expert system 16 provides an output 18 display of enhanced image findings and a risk assessment for diagnosing certain medical conditions. In an exemplary embodiment, the image acquisition system 20 may be an X-ray system providing image data of X-ray radiographs of a patient. The risk assessment is based on both the image data 12 and the non-image data 14. In an exemplary embodiment, the enhanced image findings may be CAD image findings. In an exemplary embodiment, the output 18 may optionally recommend next and/or follow-up steps for proceeding with patient care for a particular patient. In an exemplary embodiment, a user interface 22 may be coupled to the image acquisition 20 for controlling operation of the image acquisition system 20. In an exemplary embodiment, a user interface 24 may be coupled to the expert system 16 for controlling operation of the expert system 16.

The expert system 16 provides for automatic knowledge-based analysis of image and non-image information. The expert system 16 combines knowledge-based analysis for detecting X-ray radiographic image patterns and non-image data 14. The expert system 16 detects patterns in the non-image data 14 and detects correlations between features in the image data 12 and the non-image data 14.

In an exemplary embodiment, the expert system 16 may be implemented through software, hardware, or a combination thereof. In an exemplary embodiment, the expert system 16 may be integrated into the image acquisition system 16. In an exemplary embodiment, the expert system 16 may be a stand-alone system. In an exemplary embodiment, the expert system 16 may be a fully automated system. In an exemplary embodiment, the expert system 16 may be an on-demand system that may be configurable through a user interface 24.

In an exemplary embodiment, the expert system 16 may include combinations of automated image segmentation algorithms that detect the location, shape, and contour of certain anatomical features; automated X-ray radiograph image pattern detection and classification using pattern recognition algorithms and a knowledge base of medical condition and non-medical condition radiographic findings; a rule-based or learning-based system (e.g., neural networks, support vector machines, genetic algorithms, combinations thereof); a statistical based system (e.g., Bayesian, maximum likelihood, maximum entropy). In an exemplary embodiment, the expert system 16 may use the non-imaging data 14 to customize parameter selections for image-based analysis algorithms.

In an exemplary embodiment, the expert system 16 may be configured to make non-medical recommendations for each patient. For example, at a border entry screening site the system may be configured to recommend whether the screened individual should be admitted/re-admitted, admitted/re-admitted with recommended follow-up or monitoring, or denied entry. In an exemplary embodiment, the expert system 16 may also retrieve previous results of expert system analysis of the patient data, or previous tuberculosis screenings by a physician, and recommend follow-up questions. For example, “were previous findings (both tuberculosis and non-tuberculosis) resolved, or was appropriate treatment or follow-up completed?”

In an exemplary embodiment, the EMR may include image data and non-imaging data for each individual patient. Steps for building, modifying, and updating the EMR include acquiring the image data and non-image data. The data in the EMR may be acquired in any suitable manner, including those used for generating conventional electronic medical records. For example, data may be entered manually or transmitted through wired or wireless communications links and/or networks. The data in the EMR may be updated as new data becomes available. In general, EMR data acquisition is achieved by digitizing or summarizing the data in a manner that permits it to be stored in a computer-readable medium.

In an exemplary embodiment, the non-image data may include the patient's medical history, symptoms, clinical examination results, test results, risk factors, exposure to infectious medical conditions, physiological data, histopathological data, genetic data, pharmacokinetic data, or any combination thereof.

The image data 12 and non-image data 14 may be transmitted to the expert system 16 through a wired or wireless communications interface. The EMR 30 may be coupled to the expert system 16 through a wired or wireless communications interface using a local area network (LAN) or a wide area network (WAN). The wireless communications interface may be implemented through a wireless communications protocol.

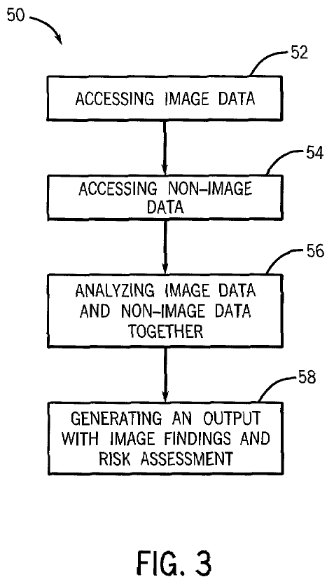

FIG. 3 illustrates a flow diagram of an exemplary embodiment of a method 50 of diagnosing a medical condition in a patient. The method 50 includes accessing image data from an image acquisition system at step 52. The method 50 further includes accessing non-image data at step 54. The combination of image data and non-image data are analyzed together at step 56. An output is then generated with image finding and a risk assessment for diagnosing a medical condition. At step 56, analysis may be performed on the data, such as to associate elements of the data with one another, as well as potentially with other data not strictly relating to the individual patient. Thus, the analysis may include consideration of additional data for populations of patients, known information relating to conditions and disease states, known information relating to risk factors for medical conditions, and so forth.

In an exemplary embodiment, a computer implemented method of diagnosing a medical condition in a patient comprises accessing image data of the patient generated by an image acquisition system; accessing non-image data of the patient; analyzing the image data and the non-image data to determine the prevalence of pre-determined patterns of interest in the image data and the non-image data; and presenting analysis results of the image data and a risk assessment of the medical condition in the patient to a user for determining a diagnosis.

In an exemplary embodiment, a computer implemented method of diagnosing a medical condition in a patient based on an electronic medical record, the method comprises accessing diagnostic image data from the electronic medical record of the patient; accessing non-image data from the electronic medical record of the patient; analyzing the image data and the non-image data to determine the prevalence of pre-determined patterns of interest in the image data and the non-image data; and presenting analysis results of the image data and an assessment of the risk of the medical condition in the patient to a user for determining a diagnosis.

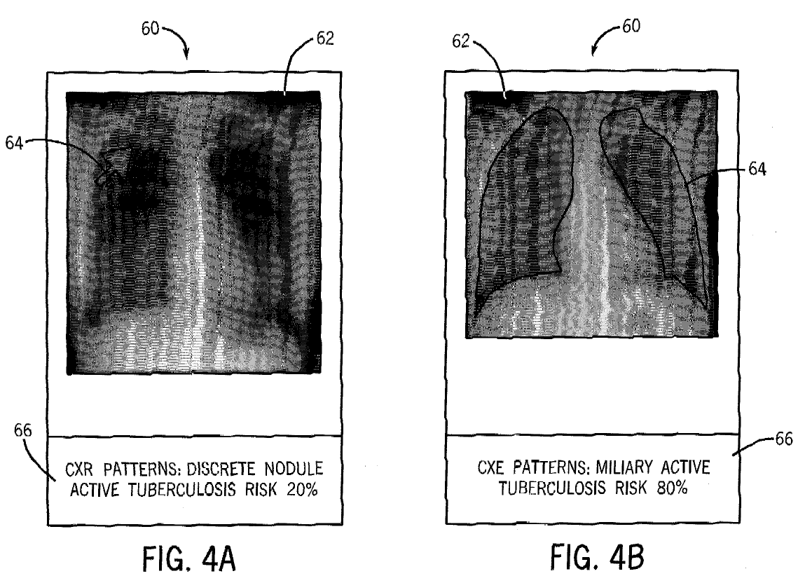

FIGS. 4A and 4B illustrates examples of outputs 60 of the exemplary system and method of the present disclosure. FIG. 4A is a schematic diagram of an exemplary embodiment of an output 60 display of a system and method of diagnosing a medical condition in a patient. The output 60 display includes a chest X-ray radiograph image 62 with detected patterns and findings indicated to the user by a visual indicator or annotation 64 on the display. For the embodiment shown in FIG. 4A, the output 60 display is a chest X-ray radiograph 62 with an indicator 64 showing a discrete nodule on the radiograph. The output 60 display also includes written text 66 of an assessment of the risk or probability of the patient having a certain medical condition, such as tubercluosis (active or past) along with further classification of type of tuberculosis. The active tuberculosis risk is listed as 20%. FIG. 4B is a schematic diagram of an exemplary embodiment of an output 60 display of a system and method of diagnosing a medical condition in a patient. The output 60 display includes a chest X-ray radiograph image 62 with detected patterns and findings indicated to the user by a visual indicator or annotation 64 on the display. For the embodiment shown in FIG. 4B, the output 60 display is a chest X-ray radiograph 62 with an annotation 64 showing a miliary pattern on the radiograph as outlines of the lungs. The output 60 display also includes written text 66 of an assessment of the risk or probability of the patient having a certain medical condition, such as tubercluosis (active or past) along with further classification of type of tuberculosis. The active tuberculosis risk is listed as 80%.

These visualizations and displays 60 are also subject to variations, such as for preferences in the manner in which images are displayed, the manner in which particular tissues are designated, highlighted, annotated, and so forth. Similar analysis techniques and reads may be performed by computer algorithms for detection, segmentation, and identification of particular tissues, particularly those that might be indicative of disease states.

Several embodiments are described above with reference to drawings. These drawings illustrate certain details of exemplary embodiments that implement the systems, methods and computer program products of this disclosure. However, the drawings should not be construed as imposing any limitations associated with features shown in the drawings. This disclosure contemplates systems, methods, and computer program products on any machine-readable media for accomplishing its operations. As noted above, the embodiments may be implemented using an existing computer processor, by a special purpose computer processor incorporated for this or another purpose, or by a hardwired system.

An exemplary system for implementing the overall system or portions of the system might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

Certain embodiments are described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Certain embodiments may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communications protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

As noted above, embodiments within the scope of the included computer program products comprising machine-readable media for carrying or having machine-executable instructions or data structures stored thereon. Such machine-readable media may be any available media that may be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which may be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machine to perform certain functions or groups of functions.

While the disclosure has been described with reference to various embodiments, those skilled in the art will appreciate that certain substitutions, alterations and omissions may be made to the embodiments without departing from the spirit of the disclosure. Accordingly, the foregoing description is meant to be exemplary only, and should not limit the scope of the disclosure as set forth in the following claims.

What is claimed is:

1. A system of diagnosing a medical condition in a patient, the system comprising:
an input of image data of the patient;
an input of non-image data of the patient;
an expert system for analyzing the image data and the non-image data to determine the prevalence of patterns in the image data and the non-image data; and
an output of the analysis results and an assessment of the risk of the medical condition in the patient.

2. The system of claim 1, wherein the input of image data is acquired from an exam of the patient on a portable X-ray image acquisition system.

3. The system of claim 1, wherein the image data is digitally created from direct digital radiography.

4. The system of claim 1, wherein the image data is digitally created from computed radiography.

5. The system of claim 1, wherein the image data is digitally created from a digitized X-ray film.

6. The system of claim 1, wherein the input of image data is acquired from a dual energy exam of the patient, wherein at least one image is acquired from at least one view.

7. The system of claim 1, wherein the input of image data is acquired from a tomosynthesis exam of the patient, wherein at least one image is acquired from at least one view.

8. The system of claim 1, wherein the input of image data includes image data acquired from a previous exam.

9. The system of claim 1, wherein the non-image data includes patient history, symptoms, test results, risk factors, exposure to infectious medical conditions, physiological data, histopathological data, genetic data, pharmacokinetic data, or any combination thereof.

10. The system of claim 1, wherein the non-image data includes previous expert system results.

11. The system of claim 1, wherein the non-image data is input manually through a user interface.

12. The system of claim 1, wherein the non-image data is input directly from an electronic medical record.

13. The system of claim 1, wherein the non-image data is updated from a previous expert system analysis of patient data.

14. The system of claim 1, wherein the expert system is configurable through a user interface to perform analysis for every patient or only on-demand for certain patients.

15. The system of claim 1, wherein the expert system combines automated knowledge-based analysis of image data and non-image data.

16. The system of claim 1, wherein the output includes images with detected findings indicated by visual annotations on the display.

17. The system of claim 16, wherein the output includes a risk assessment with a probability of the patient having a medical condition.

18. The system of claim 17, wherein the output includes next steps for proceeding with patient care.

19. A computer implemented method of diagnosing a medical condition in a patient, the method comprising:
accessing image data of the patient generated by an image acquisition system;
accessing non-image data of the patient;
analyzing the image data and the non-image data to determine the prevalence of pre-determined patterns of interest in the image data and the non-image data; and
presenting analysis results of the image data and a risk assessment of the medical condition in the patient to a user for determining a diagnosis.

20. The computer implemented method of claim 19, wherein analyzing includes automatic segmentation algorithms that detect the location, shape and contour of anatomical features.

21. The computer implemented method of claim 20, wherein analyzing includes automatic image pattern detection and classification using pattern recognition algorithms and a knowledge-base of radiographic findings of medical conditions.

22. The computer implemented method of claim 21, wherein analyzing includes rule-based analysis of non-image data in conjunction with the analysis of radiographic findings from automatic image pattern detection and classification using pattern recognition algorithms and a knowledge-base of radiographic findings of medical conditions.

23. The computer implemented method of claim 22, wherein analyzing includes non-image data to customize parameter selections for image based analysis algorithms.

24. A computer implemented method of diagnosing a medical condition in a patient based on an electronic medical record, the method comprising:
accessing diagnostic image data from the electronic medical record of the patient;
accessing non-image data from the electronic medical record of the patient;
analyzing the image data and the non-image data to determine the prevalence of pre-determined patterns of interest in the image data and the non-image data; and
presenting analysis results of the image data and an assessment of the risk of the medical condition in the patient to a user for determining a diagnosis.

25. A computer-readable storage medium having a set of instructions stored thereon for execution by a computer, the set of instructions comprising:
a routine for accessing image data;
a routine for accessing non-image data;
a routine for analyzing the image data and the non-image data; and
a routine for visualizing results of the analysis of the image data and the non-image data.
